(19) United States
(12) Patent Application Publication
(10) Pub. No.: US 2010/0003741 A1
(43) Pub. Date: Jan. 7, 2010

(54) Integrated power plant, sewage treatment, and aquatic biomass fuel production system

(76) Inventor: Howard A. Fromson, Stonington, CT (US)

Correspondence Address: ALIX YALE & RISTAS LLP, 750 MAIN STREET, SUITE 1400, HARTFORD, CT, 06103, US

(21) Appl. No.: 12/217,012

(22) Filed: Jul. 1, 2008

(51) Int. Cl. C12P 1/00; C12M 1/00

(52) U.S. Cl. 435/262.5; 435/289.1; 435/292.1

(57) ABSTRACT

Two waste products, nitrate-rich sewage wastewater and power plant CO₂ emissions, are combined and converted into a renewable, biomass energy source, which supplies the fuel to the power plant. The power plant, wastewater treatment facility, and biomass growth unit are preferably located on one site and arranged for convenient transfer of the CO₂ and wastewater to the biomass growth unit; harvesting, processing and return of biomass from the growth unit as fuel to the power plant; and discharge of the de-nitrated wastewater into the same body of water used as the heat sink by the power plant, e.g., a lake, river, or sound. The present invention thus provides an integrated approach to minimization of CO₂ emissions and nitrate discharge while achieving improved efficiency in the generation and harvesting of the biomass.

### BACKGROUND

The present invention relates to the amelioration of problems associated with some of the most fundamental activities of modern society: the generation of sewage, the generation of carbon dioxide emissions from combustion processes, and the growing demand for fuel.

The growth of the global economy has created increasing demands for energy and caused significant stresses on the environment. The consumption of fossil fuels to meet energy demands has resulted in a growing level of carbon dioxide (CO₂) in the atmosphere. It is widely accepted that increased CO₂ levels result in an increased atmospheric temperature (a phenomenon generally referred to as global warming). This temperature increase causes climate in all regions of the planet to change, with the potential for catastrophic consequences if the CO₂ level continues to rise. As urban centers grow, the volume of wastewater that must be processed by sewage treatment facilities also grows. The discharge from these treatment facilities is typically rich in nitrates. Ultimately these nitrates are discharged into the seas and oceans. They are then available as a nutrient that contributes to uncontrolled algae growth with deleterious effects on the aquatic ecosystem.

It has been previously suggested that generation of power can be combined with the controlled growth of a biomass that is ultimately rendered useable as a fuel source. The CO₂ produced by the combustion process is delivered to a site where algae or a similar aquatic plant can utilize the CO₂ and grow via photosynthesis. The biomass is harvested and utilized as a fuel for power generation.

It has also been previously suggested that aquatic algae or like biomass can be used in the treatment of nitrogen rich wastewater. After initial separation of solids in the treatment of municipal sewage, the wastewater is used as a medium for growing the biomass. The nutrient nitrates are removed from the wastewater by the aquatic algae or like biomass during photosynthesis.

### SUMMARY OF THE INVENTION

The present invention is directed to a novel method and system of addressing three fundamental needs of present day society. The inventive method simultaneously addresses the reduction of nitrates in the discharge of sewage treatment plants, the reduction of carbon dioxide emissions from the combustion processes used to generate power, and the production of fuel to meet growing energy demands. The inventive concept is especially suited to large urban areas where there are high demands for energy and large volumes of sewage wastewater.

According to the present invention two waste products, nitrate-rich sewage wastewater and power plant CO₂ emissions, are combined and converted into a renewable, biomass energy source, which supplies the fuel to the power plant.

The power plant, wastewater treatment facility, and biomass growth unit are preferably located on one site and arranged for convenient transfer of the CO₂ and wastewater to the biomass growth unit; harvesting, processing and return of biomass from the growth unit as fuel to the power plant; and discharge of the de-nitrated wastewater into the same body of water used as the heat sink by the power plant, e.g., a lake, river, or sound.

The present invention thus provides an integrated approach to minimization of CO₂ emissions and nitrate discharge while achieving improved efficiency in the generation and harvesting of the biomass. By providing a growth environment rich in both nitrates and CO₂, the photosynthetic mechanism by which biomass growth takes place is significantly more efficient than the growth rate in environments where the level of only one nutrient is elevated. Thus two entities that are otherwise perceived to be undesirable waste products, nitrates and CO₂, are now not only prevented from contaminating the environment but are utilized as raw materials in a highly efficient biomass production process providing a source of fuel for the generation of power.

The discharge wastewater from a sewage treatment facility is delivered to a location where a power generation plant is generating energy via combustion. The CO₂ produced in the power generation is delivered into the nitrate rich wastewater. Thus a growth medium for a biomass is created with highly elevated levels of two nutrients. The biomass is harvested and utilized as a fuel source for the power generation plant. The invention is thus best suited for urban areas with high sewage outfalls and high energy demands.

In a preferred embodiment, the wastewater discharge from a sewage treatment plant is delivered to a biomass growth unit with a structure whereby the flow is directed along a lengthy circuitous channel. This design of the channel is of sufficient length and size to allow for the necessary dwell time for biomass growth to take place. The CO₂ from the exhaust of the power generation process is delivered to the wastewater stream where it is captured. A biomass source with sufficient levels of algae to serve as a feedstock is also introduced into the stream at the entry point. The continuous delivery of the sewage wastewater causes the stream to flow along the channel during which time the growth of the biomass proceeds in the nutrient rich medium. As the flow proceeds, the CO₂ and the nitrates are depleted from the stream while utilized in the photosynthetic growth of the biomass. At the exit end of the channel the biomass is harvested and the wastewater stream can be discharged to the environment with considerably reduced levels of nitrates and CO₂.

Some portion of the harvested biomass can be directed back to the entry point to serve as the required feedstock. The balance of the harvested biomass can be rendered into a useable fuel source for the power plant. In an additional embodiment, some portion of the energy produced by the power plant can be utilized to provide light that enables the photosynthetic process to proceed during overnight hours of operation when power demand declines.

In addition to the increased biomass generation rate, the present invention has other advantageous features. The CO₂ does not need to be distributed over a large area, such as a pond. The area of CO₂ infusion can be localized to the entry of the channel where the sewage effluent is introduced. The flow carries the CO₂ and the nitrate rich water through the channel. Additionally, the flow naturally serves as a means to deliver the accumulated biomass to the harvesting operation at the end of the channel.

### BRIEF DESCRIPTION OF THE DRAWING

An embodiment will be described with reference to the accompanying drawing, in which:

FIG. 1 is a schematic of an integrated power plant, sewage treatment, and aquatic biomass fuel production system situated adjacent to a river;

FIG. 2 is a diagrammatic representation of the process steps for producing biomass with enhanced nutrients and carbon dioxide;

FIG. 3 is a plan view of the preferred biomass growth unit in which algae or the like grow rapidly while carried along in a flow of effluent from the sewage treatment facility in a long, circuitous channel; and

FIG. 4 is a section view along lines 4-4 of FIG. 3, showing the preferred features at the infeed region of the biomass growth unit.

### DETAILED DESCRIPTION

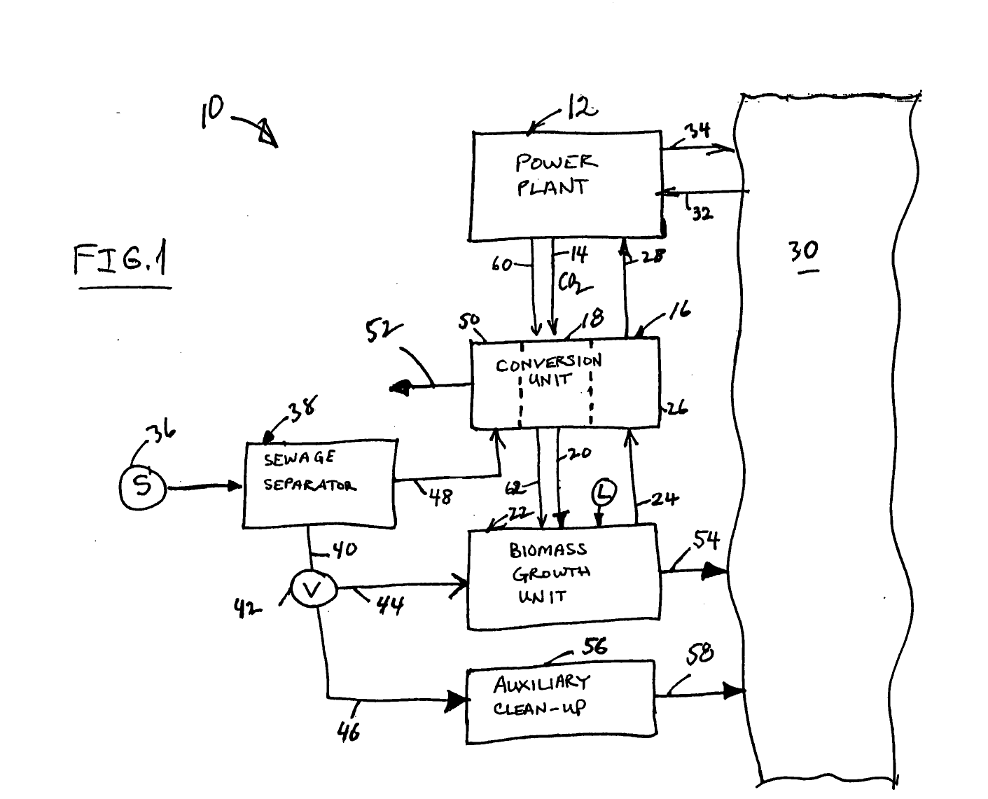

FIG. 1 is an overview of the system 10 in which a power plant 12 generates CO₂ as a component of the combustion exhaust, with the CO₂ selectively removed and delivered via line 14 to a conversion unit 16. The conversion unit has a section 18 in which the hot CO₂ gas can give off its heat for use in other process steps to be described below. The CO₂ then exits the conversion unit 16 and passes through line 20 to the bio-mass growth unit 22. Any undesirable contaminants such as sulfur-containing compounds, heavy metals or the like can be filtered out of the CO₂ stream prior to introduction into the biomass growth unit 22.

In the biomass growth unit 22, rapidly-reproducing biomass such as algae, water hyacinths or the like can grow rapidly via photosynthesis in the presence of the CO₂ gas. The bio-mass material is harvested and delivered via line 24 to the conversion unit 16, where in section 26, the bio-mass is dewatered and further converted into a fuel which is delivered via line 28 to the power plant. The power plant is typically located near a body of water 30, where cooling water for equipment is drawn via line 32 and water from condensers, or the like, of the turbine generator units is discharged via line 34 back into the body of water.

In an innovative feature of the present invention, a sewage separator 38 is integrated with the power plant 12 and biomass growth unit 22. A source 36 of sewage enters a separation unit 38 where the liquid effluent is drawn off via line 40 through control valve 42 and may be split into waste water lines 44 and 46. The solids from the separation unit 38 are delivered via line 48 to section 50 of the conversion unit 16. The solids can be converted to a form which is removed from the site at 52. The separated liquid from line 40 still contains nitrates which cannot be discharged directly into the body of water 30. Instead, the liquid waste water in line 44 is delivered to the infeed of biomass growth unit 22. The nitrates in this liquid provide nutrients for enhancing the growth of the biomass. The source 36 may generally be located on, near or a significant distance away from the premises of the sewage separator 38.

It should be appreciated that the functions performed in the conversion unit 16 can be performed independently, at separate locations.

Preferably, the high nutrient concentration in the liquid of line 44 and the plentiful supply of CO₂ from line 20 promote rapid, bloom growth of the algae or like organisms in the biomass growth unit 22. Ideally, by the time the liquid has passed completely through the growth unit 22, most of the CO2 and nitrates have been picked up in the photosynthesis process and, at the harvesting of the biomass, the remaining liquid effluent has a sufficiently low level of CO2 and nitrates that it can be discharged at 54 directly into the body of water 30. In the event that the liquid 40 from the separation unit 38 exceeds the capacity of the biomass growth unit 22, some of the flow can be diverted to line 46 and nitrates removed in a conventional manner in an auxiliary clean-up unit 56 before discharge via line 58 into the body of water.

Additionally, hot combustion exhaust from the power plant 12 can be captured and delivered to the conversion unit 18 via line 60. The heat from the exhaust can be delivered via line 62 and employed to heat the biomass growth unit 22 as needed. It is well known that over temperature ranges that allow reproduction, reproduction of algae and like organisms is inhibited as temperature decreases. Thus, the disclosed method provides an efficient approach to maintaining a steady reproduction rate in the biomass growth unit 22, especially during colder seasons.

The foregoing represents a significant integration of two sources of waste, the sewage and the power plant CO₂ emissions, with a biomass growth and processing unit, whereby the CO₂ and nitrates are converted into a bio-fuel that is reused in the power plant, while the biomass growth unit also effectively removes nitrates from the waste water so that it can be directly discharged into the environment. Further, by utilizing the excess heat from the power plant to help keep the biomass growth unit at a constant temperature while the nitrate-rich effluent and CO₂ emissions constantly flow, the rate at which biomass propagation occurs is enhanced.

It should be appreciated that the sewage and power plant emissions are unusually fit for integration into such a system. Due to the continuous nature at which sewage and power production facilities run, the biomass growth unit can be constantly fed with nitrates and CO₂, thus enabling continuous bio-fuel production.

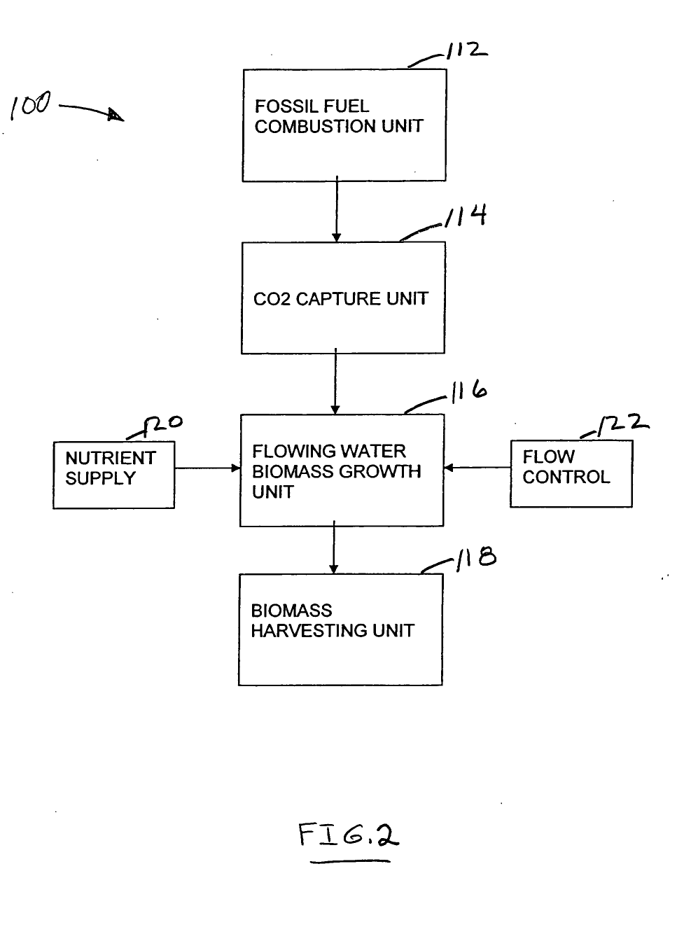

With reference to FIG. 2, a preferred biomass growth system 100 includes an exhaust line from the combustion unit 112 containing CO₂, which is captured at 114 and delivered to a flowing water biomass growth unit 116. As will be described in greater detail below, the growth unit is an outdoor water maze in which an aquatic plant material such as algae or water hyacinths is introduced at the upstream end, grows at a rapid rate while flowing through the maze, and is harvested at unit 118 at the end of the maze. The biomass growth is driven by the photosynthesis process in a flowing medium having nutrients from the supply 120 of wastewater from the sewage treatment plant. The volume or mass of harvestable biomass material at the discharge end of the growth unit can be increased or decreased by means of the flow control 122 of the water through the growth unit 116.

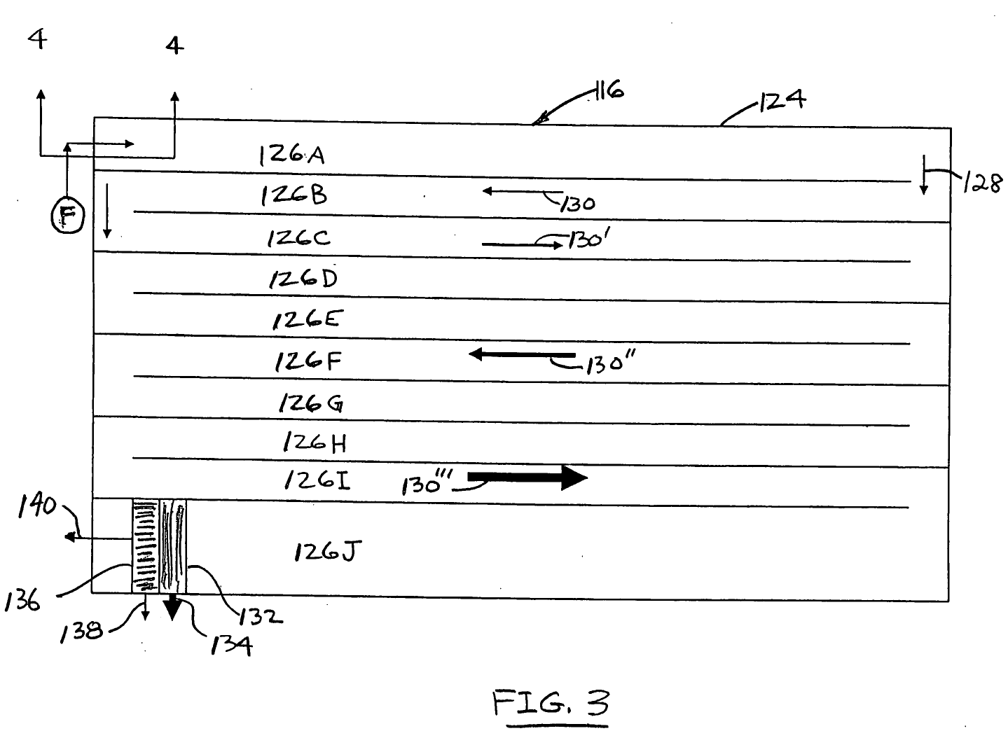

FIG. 3 shows the biomass growth unit 116 in the form of an open-top, rectangular structure 124, built into or above ground, having a plurality of internal walls arranged to provide circuitous flow channels 126A, B . . . J, fluidly connected, such as shown at 128. The channels are oriented for maximum exposure to natural sunlight. A source of wastewater inlet flow F is introduced at the entrance to the first channel 126A and is provided with an effective amount of seed algae. The water flows in the direction of the arrows 128 and 130. The increasing thickness of the arrows 130, 130′, 130″, and 130′″ indicate the density or concentration of algae material in the water. It is known, for example, that some under favorable conditions, some algae can double every hour. If all ten channels 126A-126J are of equal cross-section of flow area, and the flow is controlled to traverse one channel in one hour, a given unit volume of water and associated plant material will take ten hours to travel from the source of the inlet of channel 126A to the outlet of channel 126J. In ten hours the concentration of plant material will increase by 2¹⁰, i.e., over 1,000 times. This high concentration of biomass material is harvested on a screen or similar capture device 132 and removed at 134 for conversion at 26 and use as a bio-fuel delivered via line 28 for combustion in the power plant unit 12 (FIG. 1). Following the harvesting of the biomass material, a subsequent filter or treatment device 136 can be provided for removing residual material at 138 before the effluent is discharged at 140 (via line 54 in FIG. 1).

In one effective example, the footprint of unit 116 would be approximately one acre (43,560 sq. ft.) resulting in approximately 40,000 sq. ft. of water surface area. With ten channels, each channel would have approximately 4,000 sq. ft. of surface area. Consistent with the overall approximate one acre foot print, each channel can be 200 ft. long and 20 ft. wide, with a depth in the range of 3-5 ft. In another variation, the structure 116 can have 50 channels, each 200 ft. long, with a width of about 4 ft. each. The channel number and size are design options taking into account cost, flow speed, flow control, evaporation, sunlight penetration, etc.

Optionally, an artificial light source L, powered by the power plant, can be provided for use during off-peak, non-daylight conditions.

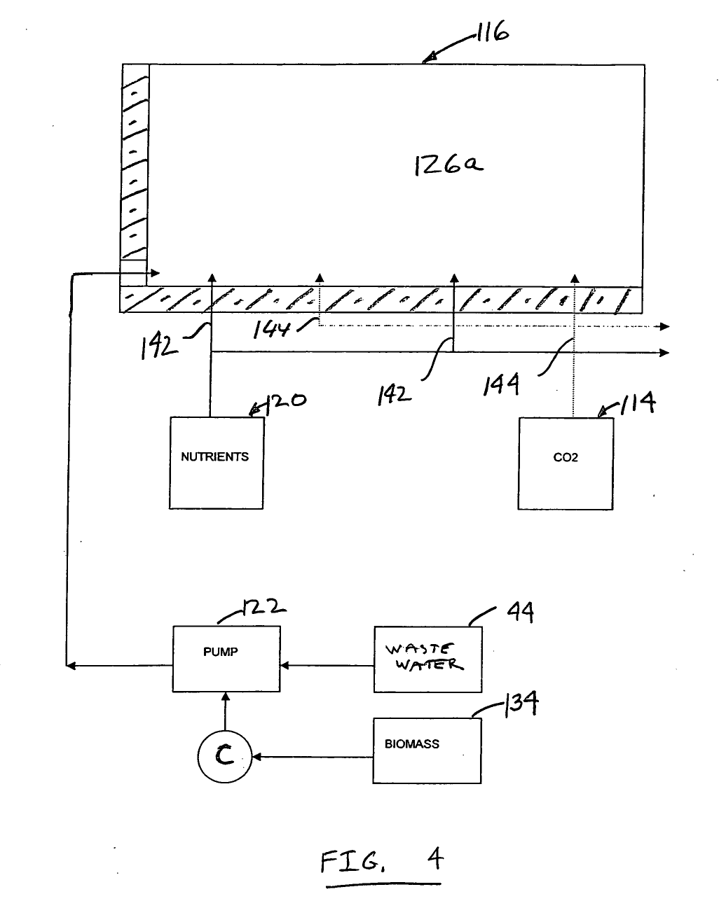

FIG. 4 is a schematic section view along line 4-4 of FIG. 3. Either through the floor or side walls of each channel, supplemental nutrient supply lines 142 can optionally deliver a controlled amount of nutrients (such as Nitrogen) from nutrient supply 120, to the extent the nutrients in the wastewater should be increased for optimization of biomass growth. Likewise, the CO₂ from the CO₂ capture unit 114 is introduced via lines 144 into the channels. It should be appreciated that means would normally be provided for controlling the rate of nutrient introduction 142 and CO₂ introduction 144, and that such rates may differ from channel to channel as the biomass grows, to accommodate the increasing volume of the biomass as it traverses the channels. Furthermore, the waste water flow rate from unit 38 via line 44 (FIG. 1) is also controlled by a variable speed pump 122, which receives an input signal from the biomass extraction device 134, such that a control signal C is sent to the pump 122.

The concentration of nutrients and CO2 should be high initially to maximize algae growth rate during the early and middle portion of the overall flow path, but as the mass of algae or like biomass approaches the end of the path for harvesting, the concentration of CO2 and nitrates in the medium should be at the practical minimum, so the effluent after harvesting can be directly discharged into the environment. This objective can be achieved by determining the difference in the mass of algae or like biomass at the infeed and harvesting locations; the quantity of nitrates and CO2 required in photosynthesis to achieve this total increase in mass; the flow volume and rate through the maze; the light intensity; and measurements of CO2 and nitrate concentrations at various points along the maze. These variables can be analytically and/or empirically related to arrive at a suitable control scheme.

As viewed in steady-state on a macro level in a community, the system achieves an absolute reduction in the quantity of carbon dioxide and nitrates. Much of the carbon dioxide and nitrates in the exhaust gas and sewage treatment wastewater delivered to the biomass growth unit, are consumed as raw material for the photosynthetic growth of a biomass and thereby converted into other, useful or harmless compounds. At the same volumetric flow rate, the effluent discharged from the biomass growth unit to the environment, contains a lower concentration of nitrates than in the wastewater delivered to the biomass growth unit. Similarly, the quantity of carbon dioxide in the effluent is discharged from the biomass growth unit to the environment, at a rate per unit time that is lower than the rate per unit time carbon dioxide is delivered to the biomass growth unit from the exhaust gas.

It should be appreciated that, as shown in FIG. 3, the flow width of each channel need not be the same. Although in a continuous operation the volumetric flow rate would be the same in each channel, the ratio of surface area to flow rate would depend on the width of the channel. This variable would be useful to accommodate the increasing concentration of biomass material, i.e., in the wide channel such as 126J, the average depth of the biomass material is closer to the surface, minimizing attenuation of sunlight due to the high concentration of the biomass material.

It should be understood that variations of the foregoing are also possible. Whereas it is preferable that the water flow be continuous from inlet to discharge, such flow could be intermittent to some extent, i.e., continual. The channels need not be parallel, but could for example be arcuate. Under certain geographic constraints, the channel could be a single, long culvert. Preferably, however, the general form of the growth unit would have a continual flow through a circuitous channeling, where the term “circuitous” means having a change in direction.

1. A method for growing biomass while simultaneously reducing the quantity of nitrates and carbon dioxide that would otherwise be available for discharge into the environment, comprising:
a) delivering wastewater containing elevated levels of nitrates, from a sewage treatment facility to an entry location of a biomass growth unit;
b) delivering carbon dioxide derived from exhaust gas of a power generation plant to said biomass growth unit;
c) providing a feed stock of biomass to the wastewater at the entry location of said biomass growth unit;
d) in the wastewater delivered to the biomass growth unit, photosynthetically growing a biomass from said nitrates and said carbon dioxide as said delivered nitrates and carbon dioxide are consumed and thereby converted into other compounds; and
e) harvesting the grown biomass.

2. The method according to claim 1, including a step f) of discharging effluent from the biomass growth unit to the environment, containing a lower concentration of nitrates than in the wastewater delivered to the biomass growth unit.

3. The method according to claim 2, wherein step f) includes discharging carbon dioxide in the effluent from the biomass growth unit to the environment, at a rate per unit time that is lower than the rate per unit time than the carbon dioxide is delivered to the biomass growth unit from the exhaust gas.

4. The method according to claim 1 wherein said biomass growth unit comprises an elongated channel containing said wastewater.

5. The method according to claim 4 wherein the biomass growth proceeds during continual flow of said wastewater through said channel.

6. The method according to claim 5 where said biomass grows to a target mass that is delivered by said flow of wastewater to a biomass harvester.

7. The method according to claim 1, wherein at least a portion of said harvested biomass is delivered as a fuel source for said power generation plant.

8. The method according to claim 1, wherein a portion of said harvested biomass is provided as said feed stock.

9. The method according to claim 1, wherein a portion of the energy produced by said power generation plant is used to provide light to facilitate growth of said biomass.

10. The method according to claim 1, wherein a portion of the energy produced by said power generation plant is used to provide heat to facilitate growth of said biomass.

11. A system for growing biomass while simultaneously reducing the quantity of nitrates and carbon dioxide that would otherwise be available for discharge into the environment, comprising:
a sewage treatment facility that generates wastewater containing elevated levels of nitrates;
a biomass growth unit exposed to sunlight and having an infeed end and a discharge end;
a wastewater delivery path from the sewage treatment facility to the biomass growth unit;
a power generating plant having a fuel combustion unit that emits an exhaust containing carbon dioxide gas;
a gas delivery path from the power generating plant to the biomass growth unit, whereby carbon dioxide from the exhaust gas is introduced into the wastewater in the biomass growth unit;
a source of feed stock of said biomass;
a feedstock delivery path into the wastewater at the infeed end of the biomass growth unit; and
a biomass harvester at the discharge end of the biomass growth unit.

12. The system according to claim 11 wherein said biomass growth unit comprises an elongated channel containing a continual flow of said wastewater from the infeed end to the discharge end.

13. The system according to claim 12 wherein the biomass grows to a target mass that is delivered by said flow of wastewater to the biomass harvester.

14. The system according to claim 11, including a conversion unit for converting said harvested biomass to a bio fuel that can be combusted in said combustion unit.

15. The system according to claim 11, wherein the source and delivery of said feedstock to the infeed of the biomass growth unit comprises a return path from a location in the biomass growth unit downstream of the infeed, to the infeed, whereby some of the grown biomass in the biomass growth is provided as said feed stock.

16. The system according to claim 11, wherein said biomass growth unit comprises a circuitous channel.

17. The system according to claim 16, wherein said biomass is delivered to a biomass harvester by said flow of said wastewater through said circuitous channel.

18. The system according to claim 11, including an artificial light source over the channel and an electrical supply path from the power plant to the light source.

19. The system according to claim 11, wherein
the power plant, wastewater treatment facility and biomass growth unit are on a single site adjacent to a body of water that provides cooling water and a heat sink for the power plant; and
a flow path is provided from the discharge end of the biomass growth unit directly to the body of water.

20. The system according to claim 11, including an artificial heat source in the biomass growth unit and a heat supply path from the power plant to the artificial heat source.
